(19) United States
(12) Patent Application Publication
(10) Pub. No.: US 2007/0191728 A1
(43) Pub. Date: Aug. 16, 2007

(54) Intrapartum monitor patch

(76) Inventor: Adnan Shennib, Dublin, CA (US)

Correspondence Address: GLENN PATENT GROUP, 3475 EDISON WAY, SUITE L, MENLO PARK, CA, 94025, US

(21) Appl. No.: 11/352,014

(22) Filed: Feb. 10, 2006

(51) Int. Cl. A61B 5/04; A61B 5/00

(52) U.S. Cl. 600/546; 128/903; 600/511; 600/549; 600/301

(57) ABSTRACT

The invention provides an integrated patch for the non-invasive monitoring of a laboring woman. The patch incorporates biopotential electrodes for sensing fetal ECG and EMG indicative of myometrial activity. The patch also incorporates a processor for extracting labor activity and fetal heart activity after filtering out maternal ECG from the composite biopotential signal present on the abdomen of the pregnant woman. The fetal monitor patch is thin, flexible, and incorporates a battery and biopotential amplifier network. In the preferred embodiment, the patch is disposable and worn continuously during labor or later stages of pregnancy. In a hospital embodiment for intrapartum monitoring, the patch wirelessly transmits fetal heart activity and myometrial activity to a bedside monitor or a remote monitoring station.

### CROSS REFERENCE TO RELATED APPLICATIONS

This application is related to the U.S. patent application entitled Disposable Labor Detection Patch, filed jointly with this application, and co-pending patent application Ser. No. 10/866,378. These applications are incorporated herein in their entirety by this reference herein.

### BACKGROUND OF THE INVENTION

1. Technical Field

The invention relates to non-invasive monitoring of vital signs of a laboring woman. More particularly, pertaining to monitoring of fetal heart rate and myometrial activity.

2. Description of the Prior Art

Techniques to monitor vital signs of a fetus and the expectant mother during labor and delivery have been developed and are widely used in clinical settings. Intrapartum monitoring provides assurance and can determine if intervention is required. Timely detection of fetal distress is important and can have a profound influence on fetal outcome. Monitoring of fetal heart rate (FHR) is particularly useful in assessing the general health of the baby, as well as the baby's vascular system in particular. Vital signs, such as average fetal heart rate, beat-to-beat rate, and variability are altered by the sympathetic and parasympathetic nervous system, and thus provide an excellent indication of the well-being of the baby. For example, the absence of variability in fetal heart rate is an ominous sign requiring further investigation and possible intervention by medical personnel.

Current fetal monitoring instruments use an ultrasonic transducer placed on the abdomen of the mother, a reflected ultrasonic wave from the heart is electronically decoded into a tone or heart rate. However, ultrasonic monitoring generally requires proper alignment of the transducer, and thus can be a challenge when considering the movement of the fetus in the uterus. Ultrasonic equipment is also expensive and consumes a large amount of power, and thus is not suitable for long-term battery-operated applications. Ultrasonic fetal monitoring also involves emissions towards the fetus with possible adverse effects if used continuously for long periods. For these and other reasons, ultrasound-based fetal monitoring has not been widely employed in ambulatory applications.

Current fetal monitors also offer an invasive option of fetal heart rate monitoring involving an electrode attached to the fetal scalp or a presenting part of the fetus. These and other invasive methods are well known as disclosed in the prior art including U.S. Pat. No. 5,431,171 to Harrison et al, and U.S. Pat. No. 6,115,624 to Lewis et al. These methods typically require the rupture of the protective amniotic sac and sufficient dilation of the cervix to insert the sensing electrode. These methods involve medical risks and require the presence of an obstetrical professional.

Obtaining fetal heart rate from fetal ECG present on the mother's abdomen is non-invasive but has many challenges. First, the fetal ECG signal is highly contaminated with the maternal ECG, which may be an order of magnitude stronger than fetal ECG signal. Second, the fetal ECG signal, being inherently weak, is easily contaminated with electromagnetic interference (EMI) present in the environment and interference from electromyogram (EMG) signals; due to muscle activity of the expectant mother. FIG. 8a shows a typical waveform of composite ECG with fetal ECG and maternal ECG features shown. The QRS complex of the fetus ECG (QRS_{f}) is typically weak as compared to the dominant mother QRS (QRSₘ). Other ECG features of maternal ECG can also be seen, including the T-wave (Tₘ). There is no place on the mother's skin at which only the fetal ECG can be obtained. However, the ratio of fetal ECG to maternal ECG can be improved substantially by placing electrodes near the fetus at the abdomen area including the back.

Prior patent application Ser. No. 10/866,378 discloses prior art systems and methods for obtaining relatively pure fetal ECG and FHR from abdominal ECG. This includes references to U.S. Pat. No. 5,123,420 to Paret, U.S. Pat. No. 5,372,139 to Holls et al, and U.S. Pat. No. 5,042,499 to Frank et al and U.S. Pat. No. 4,781,200 to Baker.

Incorporating contraction data along with FHR during labor has become the new standard of intrapartum care in maternity wards. At full term, generally defined as beyond 37 weeks of gestation, contractions become intense and regular to assist the mother in the normal delivery of the baby. The duration and intensity of contractions vary widely according to the condition and stage of pregnancy. Contraction patterns dramatically change during active labor beginning with dilation of the cervix and ending with the delivery of the baby and placenta.

When contraction patterns become consistent and regular, the mother is typically rushed to a hospital for delivery or at least advised to contact her health care provider. Detecting the occurrence of true labor is sometimes difficult for the mother. False labor, characterized by irregular contractures, sometimes occurs leading to unnecessary preparations and stress. On the other hand premature labor contractions leading to premature delivery may occur without pain or recognizable symptoms. Premature contractions may be confused with other abdominal symptoms such as intestinal cramps and backache.

Intrapartum monitors are currently used for monitoring fetal heart rate in combination with contractions. The non-invasive options typically rely on ultrasonic transducers and toco transducers (tocodynamometer). These transducers are typically held against the abdomen by a belt or a harness and connected to an external monitor to display FHR and pressure patterns during labor. The intensity and duration of a contraction is typically observed along with fetal heart rate (FHR) to assess the status of the baby and progression of labor. Maternal heart rate can also be monitored using standard ECG electrodes attached to the mother. Non-invasive methods do not always produce reliable fetal heart rate or pressure measurements, this necessitating the use of a scalp electrode or an intrauterine pressure (IUP) catheter to measure uterine pressure more accurately. The IUP catheter is introduced vaginally after the cervix is sufficiently dilated and comprises a pressure sensor at its tip for sensing uterine contractions and for relaying pressure signals to the external monitor.

Even with recent advances in electronic miniaturization and microprocessor applications, the cost and inconvenience of current instruments limit their application to specialized clinical settings, such as gynecology offices and hospitals. For home applications, portable monitor instruments can be used by the expectant mother but are generally limited to those with high-risk pregnancies.

U.S. Pat. No. 6,440,089 by Shine discloses a uterine contraction detector, shown as a desktop unit, with a method of determining the frequency of contractions, trending the frequency data, and generating a real-time graphical representation of the determined frequency.

U.S. Pat. No. 6,169,913 by Hojaiban et al. discloses an apparatus and method of sensing uterine activity by sensing changes in blood volume in the abdominal wall. A particular method disclosed involves detecting reflected light indicating its absorption by hemoglobin present in abdominal blood vessels.

Research has demonstrated that labor contractions can be assessed non-invasively using an electromyogram (EMG) signal. Uterine EMG also referred to as electrohysterography (EHG) characterizes uterine contractile events during pregnancy with low initial activity rising dramatically during labor.

Nathanielsz in U.S. Pat. No. 4,967,761 discloses a method of characterizing myometrial activity to distinguish true labor (term and preterm) from contractures.

U.S. Pat. No. 6,134,466 to Rosenberg discloses a method and system, shown as desktop apparatus, for detecting EMG signals by analyzing the average frequency of each contraction and indicating true labor when the last discriminant exceeds a threshold value.

U.S. patent application 2005/0267376 discloses a maternal-fetal monitoring system used for all stages of pregnancy to monitor fetal heart rate and contractions, with intelligent analysis and display tools for use in clinical diagnosis.

These and other prior art instruments and methods are not only expensive and complex, but also cumbersome when considering the physical aspect and profile of these systems.

Prior patent application Ser. No. 10/866,378 discloses a fetal monitor patch integrating ECG electrodes and processor for automatic monitoring of fetal heart rate.

An object of the invention is to provide a highly integrated, non-invasive device for the combined monitoring of fetal heart rate and contractions during labor and delivery.

A further objective of the invention is to provide a low cost, disposable monitor for a laboring female.

A further objective is to provide multi vital sign sensing for women having an at-risk pregnancy.

A further objective is to develop an intrapartum monitor that minimizes supervision and intervention by medical personnel.

A further objective is to provide a real-time intrapartum monitor, integrating an indicator and allowing mobility for the mother.

A further objective is to provide a non-obtrusive fetal-maternal monitor for hospital use, with ability to monitor fetal heart rate, maternal heart rate, and the progression of labor.

### SUMMARY OF THE INVENTION

The invention provides an electronic monitor patch for the non-invasive monitoring of the baby and the mother during labor and delivery. The patch is adhered to the abdomen of an expectant mother. The patch intercepts biopotential electrodes to sense composite surface potentials present on the woman's abdomen. The patch monitors fetal heart rate (FHR) and contractions during labor or during the later stages of pregnancy. FHR is computed from sensed fetal ECG signal, which is extracted from composite ECG signal contaminated with maternal ECG. The patch also monitors myometrial activity by sensing and analyzing EMG signal patterns during labor. Filtering and signal separation is accomplished by signal processing means, as well as proper placement of the electrodes on the abdomen. In one embodiment, an upper electrode is employed to obtain a relatively pure maternal ECG signal that is used for detecting maternal heart rate and cancellation of the maternal component from the composite ECG signal obtained from lower electrodes.

The monitor patch is thin, flexible, and also incorporates biopotential amplifiers, a processor, a memory, and a battery for self-powering of the invented patch. In the preferred embodiment, the monitor patch is disposable, and thus discarded after a single use or upon delivery of the baby. The monitor patch is suited for intrapartum monitoring or long-term wear at home, particularly for women having a risk of premature delivery.

Wireless transmission of real-time or recorded data to a remote monitoring station is preferable in one embodiment. In a hospital embodiment of the invention, fetal heart activity and EMG signals are sensed and wirelessly transmitted to an external monitor. The vital sign data can be sent to the external monitor directly or via an interface device that translates sent data to an electrical signal that emulates fetal heart activity and pressure signals produced by standard transducers. This emulation technique allows the invented patch to interface with widely available standard fetal-maternal monitors.

### BRIEF DESCRIPTION OF THE DRAWINGS

FIG. 1 is a frontal view of a labor monitor patch placed on the abdomen of a laboring woman, in which the patch is vertically elongated with biopotential electrodes for jointly sensing ECG and EMG representing myometrial activity;

FIG. 2 is detailed view of the vertically oriented monitor patch of FIG. 1 showing major internal components;

FIG. 3 is a cross section view of the monitor patch shown in FIG. 2;

FIG. 4 is a detailed, cross section view of a section of the sensor patch of FIG. 2, showing the various layers including a metal foil layer;

FIG. 5 shows a rectangular embodiment of the monitor patch having three electrodes;

FIG. 6 shows a five electrode embodiment placed on the abdomen of an expectant mother with EMG sensing for labor activity monitoring and ECG sensing for fetal and maternal heart rate monitoring;

FIG. 7 is a schematic diagram of the electronic assembly within the monitor patch, showing audible and visual indicators;

FIG. 8a shows a composite ECG signal combining fetal QRS and maternal QRS components;

FIG. 8b shows an extract QRS complex of the fetal ECG;

FIG. 9 shows a monitor patch placed on the side of the abdomen;

FIG. 10 shows an embodiment of the intrapartum monitor patch having five biopotential electrodes;

FIG. 11 shows a labor monitor patch having a wireless interface to standard fetal-maternal monitor instrument, with a wireless interface device in proximity to the patch device;

FIG. 12 shows a block diagram of the wireless interface device with a wireless receiver and signal conditioner to provide simulated signal to standard fetal-maternal monitoring instrument; and

FIG. 13 shows the invented patch equipped with acoustic transducers for transferring vital sign data acoustically over the telephone.

### DETAILED DESCRIPTION OF THE INVENTION

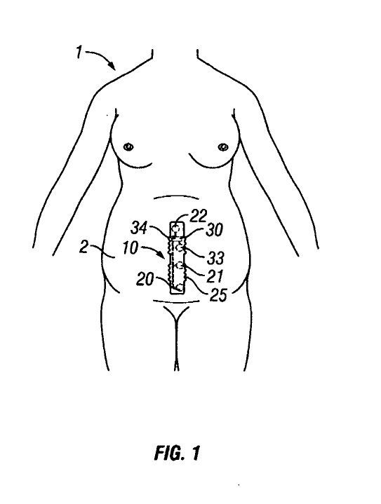

The invention, shown in various embodiments of FIGS. 1-13, is a non-invasive fetal-maternal monitor 10 in the form of a patch placed on the abdomen area 2 of a pregnant woman 1. The patch device 10 is thin and flexible for unobtrusive continuous wear during labor and delivery or later stages of labor.

Referring to the embodiment of FIGS. 1-3, the patch device 10 comprises biopotential electrodes, 20, 21, and 22 for sensing jointly ECG and EMG signals present on the abdomen of a pregnant woman. The device 10 also comprises an electronic assembly 30, including a biopotential amplifier 31, a processor 32, and a power source 33. The processor 32 receives biopotential data obtained from an analog-to-digital converter 36. The power source 33 in the preferred embodiment is a primary battery having long shelf life.

The monitoring of fetal heart rate jointly with contraction events provided by the invention is clinically significant. Normal heart rate generally suggests adequate oxygenation for the fetus from the mother's bloodstream. A typical FHR pattern is to slow slightly during a contraction, and rise again when the contraction ends. Abnormal variations in heart rate can indicate decreased oxygen in the blood and tissues of the fetus, which can lead to potential damage to the baby. Patterns that can cause concern include abnormally fast or slow heartbeat, a heart rate pattern that takes a long time to return to normal after a contraction (prolonged deceleration), a heart rate that slows late in the contraction and stays slow (late deceleration), or a heart rate that does not respond to contractions (no variability). These patterns, when detected require closer monitoring of the baby, further testing, or medical interventions. The invented patch allows for the joint FTH/contraction monitoring through non-invasive sensing of ECG and EHG signals present on the abdomen of the pregnant woman. The invented patch is highly integrated including an electronic assembly for sensing and signal separation.

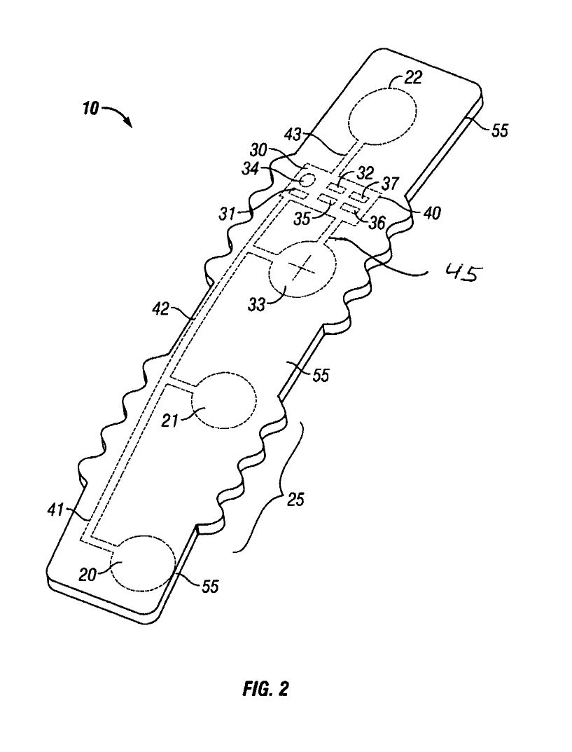

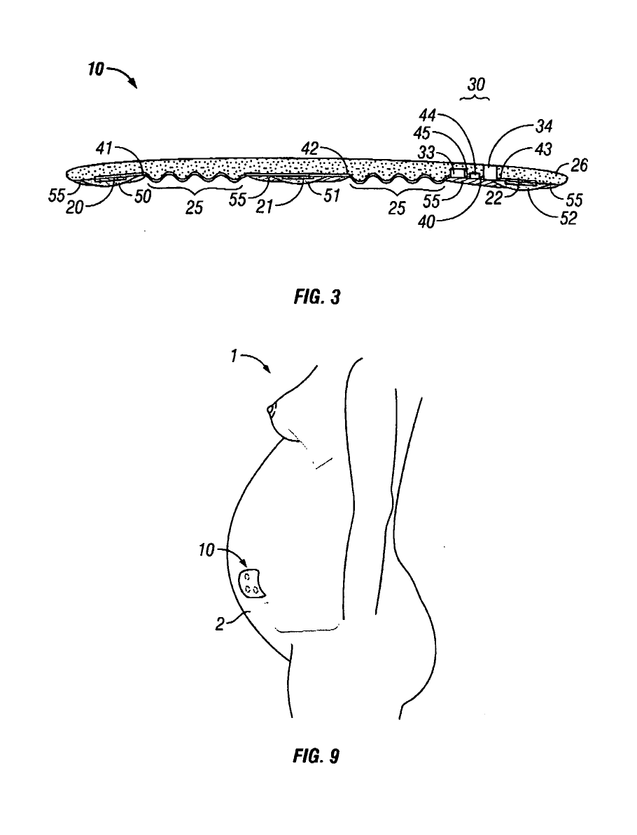

In a more detailed view of the device shown in FIGS. 2-5, the electronic assembly 30 is mounted on a flexible circuit substrate 40, with trace extensions 41, 42, 43, and 45 connecting the electronic assembly 30 to electrodes 20, 21, 22 and the power source 33, respectively. Conductive adhesive films 50, 51, and 52 cover metal electrodes 20, 21, and 22, respectively. Conductive adhesive films 50, 51, and 52 contact the skin directly to conduct surface potentials to the amplifier 31. A non-conductive adhesive 55 provides an overall adhesive to secure the patch device 10 to the woman's body. The device 10 also comprises a thin substrate 26 (FIGS. 3-5) that provides structural support. The substrate 26 is made of soft flexible sheath material, such as polyurethane, cotton, or other material used in medical patch applications. The thickness of the patch device 10 is preferably in the range of 2 to 5 mm, but preferably no more than 6 mm.

The patch assembly 10 may comprise as few as two electrodes or as many as seven or more electrodes, depending on the desired application. Two to three electrodes are sufficient for basic monitoring applications in a small package for long term home monitoring applications. Additional electrodes (discussed below) and sensors (not shown) can be incorporated to determine additional vital signs, such as temperature and oxygen saturation levels. Fetal heart rate is extracted from a composite ECG biopotential signal (FIG. 8A) present on the abdomen of a pregnant woman. The extraction of fetal ECG (FIG. 8B) from the composite ECG is disclosed in more detail in patent application Ser. No. 10/866,378 by the same inventor, which is incorporated herein in its entirety by this reference. A major advantage of the invention is the incorporation of biopotential amplifiers to amplify biopotential signals closer to the source, a technique known to improve signal-to-noise ratio. This is in stark contrast to conventional monitoring systems that connect skin electrodes to external amplifiers via cables. Another advantage of the invention is that the electrodes within the patch are specially prearranged to enable quick and easy placement of the inverted device on the body of a pregnant woman.

FIGS. 1-4 show an elongated patch arranged in a vertical electrode configuration. FIG. 5 shows an alternate three-electrode configuration, where the patch is rectangular in shape, having an upper electrode (Eᵤ), and two lower electrodes, E_{R}, E_{L} for placement on the right and left sides of the woman's lower abdomen.

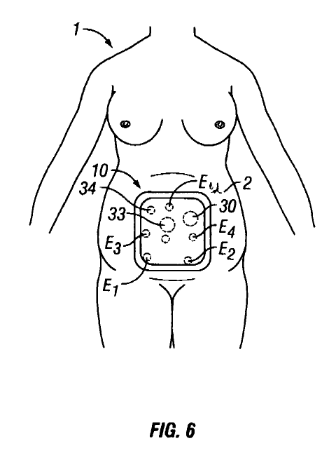

FIG. 6 shows a five-electrode embodiment, having an upper electrode Eᵤ and four abdominal electrodes E₁, E₂, E₃, and E₄, for the combined monitoring of ECG and EHG. The combined measurement using the same set of electrodes provides an integrated electronic solution for vital sign sensing without resorting to the electromechanical transducers that are used in conventional fetal monitoring systems. This results in drastic cost reductions and new disposable applications, as disclosed herein.

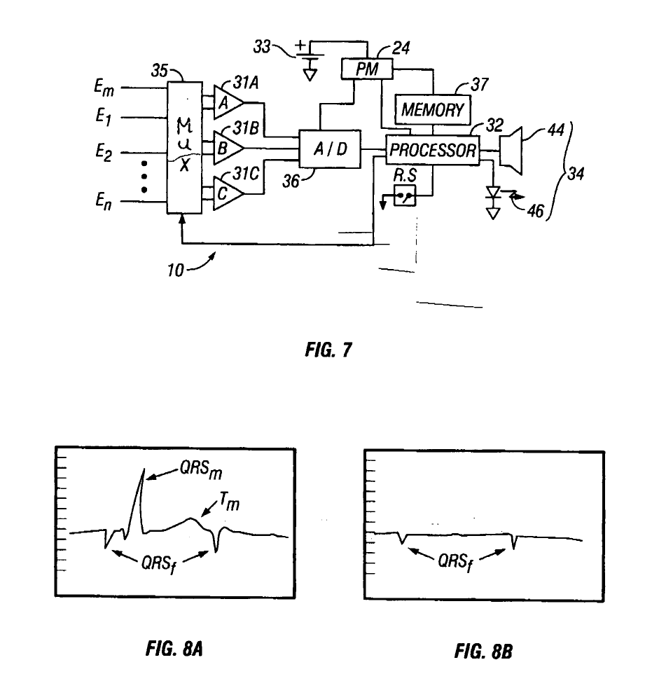

The multi-electrode configuration is also useful in applications to minimize the effects of artifacts present on the abdomen and for ensuring continuous reliable detection of EMG and ECG signals. Multiple electrodes minimize the effect of variable fetal position and movements. FIG. 7 shows a multiplexer (MUX, 35) for selecting and pairing electrodes as differential inputs to biopotential amplifiers 31A, 31B, and 31C. Because the multiplexer 35 is under the control of the processor 32, selection of electrode pairs can be dynamically performed in real-time to obtain the desired biopotential signal. Alternatively, the application of adaptive signal processing for signal enhancement and cancellation of undesired signal can be accomplished digitally with a fixed set of biopotential amplifiers. Thus, an analog multiplexer is not required.

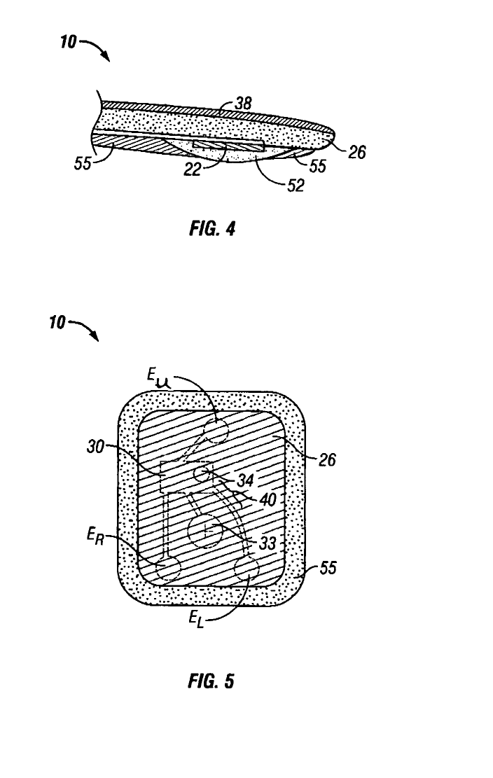

Various filtering methods are known in the field of signal processing, and particularly pertain to EMG and ECG signals. Filtering is not only necessary for removing undesired biopotential signals, such as maternal ECG and muscular EMG, to obtain fetal ECG, but also for filtering out electromagnetic interference (EMI). To minimize interference further, a metal foil 38 (FIG. 4) is preferably provided, either entirely over the substrate 26 or selectively over electronic traces and components sensitive to interference.

In an embodiment of the invention, the patch is used to detect early signs of premature contractions for mothers with risk of premature delivery. By continuously sensing EMG patterns on the abdomen of a pregnant woman, true labor and adverse contraction conditions can be detected and differentiated from ordinary myometrial contractures experienced during false labor. The intelligent patch of the invention may be programmed to detect and indicate the occurrence of labor once contractions occur exceed a certain rate, i.e. four times per hour. When this occurs, the patch of the invention alerts the mother via the integrated indicator 34 which may be of any form perceptible by the expectant mother. The pregnant woman can then alert her medical provider for intervention, which may include the administration of a tocolytic agent to halt or delay a premature delivery. The indicator 34 may also be used to indicate the progression of labor from an early stage through later stages. An indicator in the form an alarm transducer can be activated during an adverse condition detected by the monitor device 10. An adverse condition includes abnormal fetal heart rate, abnormal maternal heart rate, premature contraction, hyperstimulation, hypertonus, etc.

The indicator transducer 34 may be in the form of an audible transducer (44, FIG. 7), such as a buzzer or a speaker; or it may be in the form of visual display 46, such as a light emitting diode (LED) or a liquid crystal display (LCD). In the case of a visual indicator, each stage of labor can be indicated by text display or the color of an LED. For example, a green LED can be used to indicate early labor, an orange LED for first stage active labor, a red LED for second stage active labor, etc. Another example of an indicator transducer is a vibrating element for imparting tactile sensations to the mother during a stress condition. The indicators may also be used to indicate the status of the monitor, such as battery or other monitored parameters, such as fetal and maternal heart rate. The invention provides an integrated solution with sensing, biopotential amplification and signal separation, all incorporated in the patch device.

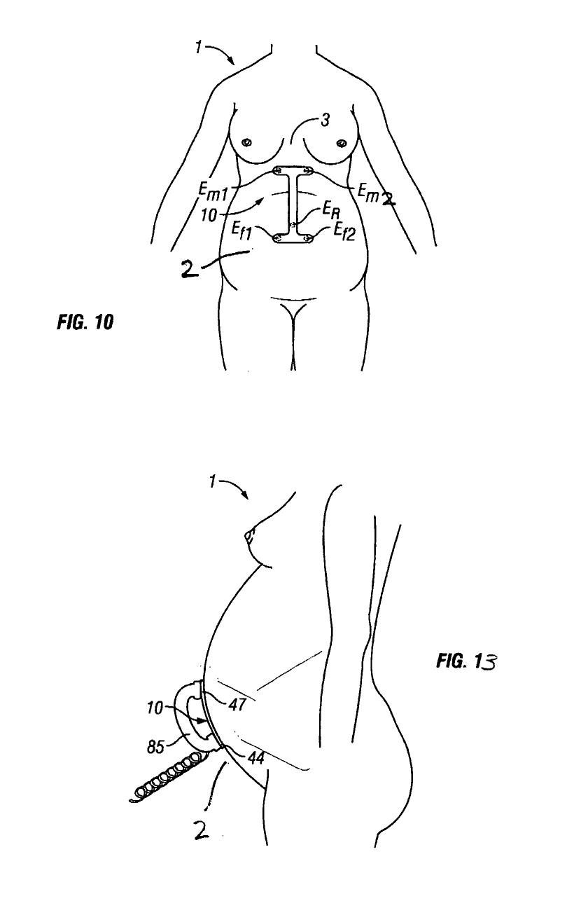

FIG. 9 shows an embodiment of the labor monitor patch device 10 placed on the side of the abdomen. Other configurations of the invented patch include five electrodes configured in an “H” format as shown in FIG. 10. In this configuration, electrodes Eₘ₁ and Eₘ₂ are used for sensing of EMG and maternal ECG. Abdominal electrodes E_{f1} and E_{f2} jointly receive EMG and fetal ECG contaminated with maternal ECG component. A reference electrode E_{R} provides reference input. The multi-electrode embodiments allow for simultaneous monitoring of contraction, fetal heart rate (FHR), and maternal heart rate (MHR), thus particularly suitable for maternal-fetal monitoring in hospitals during labor and delivery. Other electrode configurations (not shown) include patch placement on the back side of the abdomen or extending from the front to the back of the abdomen. An advantage of back placements is improved stability because the tissue is less subject to deformations, compression and movement.

In the hospital embodiment of the invention, shown in FIG. 11, it is desirable to provide a wireless link 62 from the intrapartum monitor patch 60 to an external monitor 65 to display monitored parameters sensed by the invented patch 60 on the display unit 66 or the printout 69 of the external monitor 65. The wireless link 62, shown as RF signal, allows the mother to be ambulatory during labor while providing continuous uninterrupted data for the medical staff.

EMG signals detected during contractions can be displayed by a standard fetal-maternal monitor 65 using standard toco input 67′, IUP input (not shown), and ultrasound input 68′, as shown in FIGS. 11 and 12. This is partially accomplished by providing an interface device 70 that produces an electrical signal 77 which is compatible with the signal produced by a toco transducer or an IUP transducer. The interface device 70 comprises a wireless antenna 71, a wireless receiver/decoder 72, an amplifier 72, and a signal conditioner 76 for producing an electrical signal 77 having a format and levels that emulate signals produced by standard toco or IUP sensors. The contraction signal 77 is delivered through a standard toco plug 67 which feeds into the toco input 67′ of the external monitor 65, resulting in a standard contraction display 66 and printout 69. The interface device 70 translates and correlates EMG activity, which is electrical in nature, to pressure signals (typically mm Hg), which are widely used and accepted. For example, a baseline of EMG activity at rest sensed by the invented patch can be electronically correlated to produce a baseline display of approximately 10 mm Hg on the external monitor 65 by producing the corresponding signal level into the toco input 67′. On the other hand, an intense EMG burst activity can be electronically correlated to produce a display of 80 mm Hg on the display unit 66 of the external monitor 65. Similarly, fetal heart rate (FHR) information can be sent by the wireless patch 60 for receiving by the wireless receiver/decoder 72 and for processing by the FHR amplifier 73 and the FHR signal conditioner 74. The interface device 70 produces an FHR signal 78 that is compatible with the signal produced by an ultrasound transducer. The FHR signal 78 is delivered via an ultrasound plug 68 to ultrasound input 68′ of the external monitor instrument 65. The interface device 70 preferably comprises a visual link indicator 79 that indicates proper a wireless link with the invented patch 60 when it is detected in proximity. The interface box 70 is preferably powered by power signal supplied by the toco input 67′, ultrasound input 68′, IUP input (not shown) or other ports available at the external monitor. This allows for self-powering of the interface device 70, which eliminates the need for a battery or separate power supply for the interface device. In one embodiment, an external monitor or a personal computer using a protocol, such as Blue Tooth or 802-11, and an appropriate software application as is known in the art can be used to receive and process signals from this device.

A wireless link from the invented patch to an external monitor is highly advantageous but a cable connection is possible and within the scope of the invention, having a labor monitor patch integrating prearranged electrodes, biopotential amplifiers, and means for processing or transmitting ECG and EMG signals to a remote monitor. To provide continuous intrapartum monitoring during labor through delivery, the shape of the invented patch can be designed with access considerations to surgical intervention, such as a cesarean section.

It should be obvious to those skilled in the art of medical electronics that other connections and input arrangements for connecting the interface device or the invented patch to an external monitor in clinical setups are possible. For example, a report or data can be send directly to a wireless printer from the invented patch. It should also be obvious that the contraction and FHR data can be of any suitable format including raw ECG and EMG data and processed data ready for display on an external monitor or a printer. Furthermore, it should be obvious that the invented patch is not only suitable for monitoring labor in pregnant women but also applicable to mammals.

Relaying vital signs to an external monitor is highly advantageous. However, by incorporating indicators on the invented patch, all or part of monitoring process can be performed by observing the indicators thereon. For example, a miniature LCD can indicate vital signs directly on patch to the medical staff or the mother. Alternatively, a multicolored LED can indicate normal vs. abnormal activity as detected by the smart patch of the invention.

Mobility afforded by the invention is known to reduce stress for the mother during labor and may also shorten the duration of labor, leading to reduced distress on the mother as well as the baby. Ambulation during labor is limited with current intrapartum monitoring instruments because the mother is typically confined to the bed with sensors attached to the mother on one end and a bedside monitor on the other end. The invented patch in the wireless embodiment provides a disposable electronic alternative that is more hygienic and less prone to loss of signal compared to the conventional electromechanical sensors currently in use. The reusable sensors of conventional monitors are cumbersome to apply and require frequent cleaning and application of gel. Furthermore, movements of the mother and baby often necessitate repositioning of the sensors or adjustment of the belt. The electronic solution of the integrated patch of the invention is robust and relatively insensitive to fetal movements and positioning on the abdomen while allowing the mother mobility during labor.

After admission to a maternity ward for delivery and the initial wear of the invented patch for labor monitoring, the expectant mother may be instructed to walk, wait, or go home if contractions are found infrequent or too weak, or if the cervix is not sufficiently dilated. However, the laboring woman may be instructed to continue wearing the invented patch for non-invasive ambulatory monitoring until her condition advances to a later stage for readmission, as indicated by the invented patch. Discharging a laboring female with insufficient indications eliminates lengthy and unnecessary accommodations commonly experienced in maternity wards.

FIG. 7 is a schematic diagram that shows major components of a preferred embodiment comprising a reed-switch 39 (wireless sensor) incorporated in the patch device 10 for responding to a magnetic field from an external magnet (not shown) or programming device (not shown). In this programmable embodiment, the device can be configured with operational parameters according to the needs and condition of the expectant mother. Programming is preferably by wireless means by incorporating a wireless sensor in the patch to receive coded wireless commands from an external transmitter (not shown).

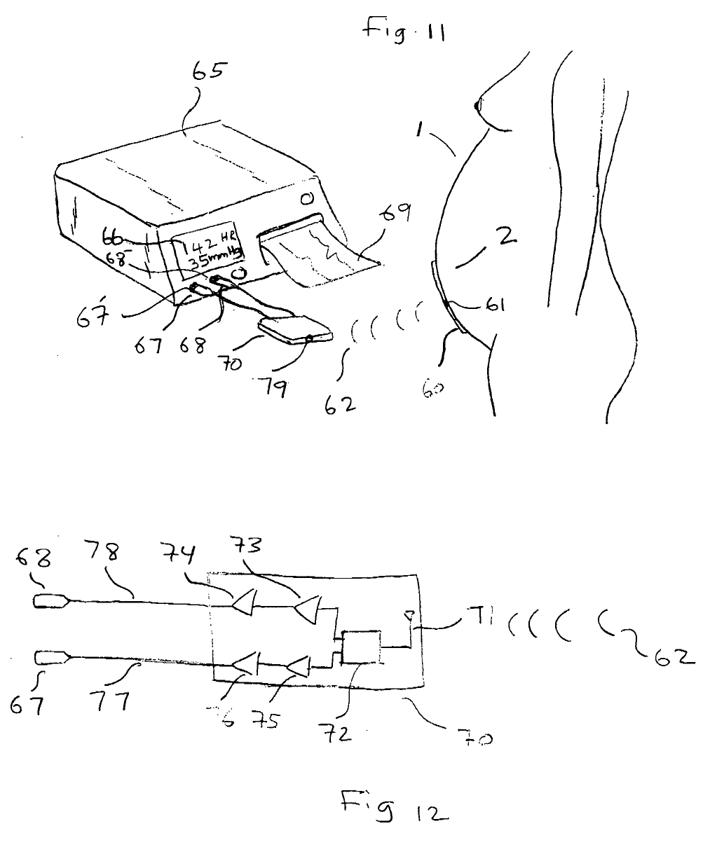

Other possible features can include the ability to store data in a memory 37 and transmit the stored data to a remote receiver, such as an external monitor 65 (FIG. 11) for display and clinical analysis by medical staff. FIG. 13 shows acoustic trans-telephonic transmission of data via an audio transducer 44 incorporated within the patch device 10 to the mouthpiece of the telephone handset 85. In this embodiment, acoustic interrogation commands from the remote unit via the earpiece of the handset can also be downloaded into the patch device 10 via the receiver audio transducer 47.

The wireless reception of commands and transmission of data may be accomplished in numerous ways and methods known in the field of remote control and wireless transmission of medical data. This includes optical, radio frequency (RF), magnetic, ultrasonic, and acoustic transmission. The programming unit can also be incorporated in the receiver unit such as within the interface device shown in FIG. 11. The programming and/or receiver unit can be in the form a desktop apparatus, a portable unit, or a handheld instrument. In one embodiment, an external monitor or a portable computer using a protocol, such as Blue Tooth, Medical Implants Communication Services (MICS), Wireless Medical Telemetry Services (WMTS), or 802.11, and an appropriate software application as is known in the art can be used to receive and process signals from the device.

For fetal monitoring of females with high risk pregnancies at home, the invented patch jointly monitors the ECG and EMG long-term. For this purpose, many design details should be incorporated for the device to function properly and reliably for extended periods of time exceeding several days. The adhesion to the abdomen skin may be designed for single-use or multiple applications. In single-use applications, the patch device is applied once for continuous wear until it is removed for its disposal. In this case, the patch is worn even during sleep and bathing. In multiple applications design, the adhesive allows for multiple removal and reapplication to the skin. In either design, the adhesive 55 incorporated in the device 10 must provide continuous reliable skin adhesion to prevent inadvertent peeling of the device from the abdomen. A biocompatible skin adhesive, such as hydrogel and like materials, has been shown to be effective in human skin applications. The ideal properties of the skin adhesive include being waterproof and air-permeable. Waterproof properties aid in the protection of the electrodes underneath from water-born contaminants. Air permeability properties allow for the healthy aeration of the tissue underneath the patch device.

To achieve longevity of operation for the patch device, various means for power conservation must be considered. This includes power management (PM) circuitry (24 FIG. 7) to shut off certain electronic components selectively when not in use. The patch device 10 may also incorporate stretchable areas 25 (FIG. 1) to allow for stretching and abdomen movements during motion, breathing, sleep, etc. The construction of the device must be durable and protective of the components within. Metal foil 38 (FIG. 4) covering the internal components and substrate 26, not only provides EMI protection, but can also aid in water proofing and overall protection. Proper patch adhesion to the skin is not only important for waterproofing purposes, but also to maintain proper electrode-skin contact throughout device wear and operation. This is important for obtaining adequate biopotential signal-to-noise-ratio.

The invention incorporates biopotential electrodes for cost, size, and integration purposes. However, other miniature sensors can be easily incorporated, including a light emitting/sensing element, a piezoelectric element, a thermistor element, and others for detecting various vital signs, including temperature, oxygen saturation, and abdominal pressure changes. These and other sensors are well known in the field of medical transducers, particularly pertaining to fetal monitoring, labor, and delivery.

The power source 33 in the preferred embodiments is a primary battery having long shelf life. However, wireless means for powering or recharging a rechargeable battery within are well known in the field of biomedical implants, including inductive coupling whereby a coil within the device (not shown) is used to receive energy wirelessly from an external coil introduced in proximity.

Although the invention is described herein with reference to preferred embodiments, one skilled in the art will readily appreciate that other applications may be substituted for those set forth herein without departing from the spirit and scope of the present invention. Accordingly, the invention should only be limited by the Claims included below.

1. An apparatus for monitoring a vital sign of a pregnant female during labor comprising:
a non-invasive monitor device worn by said pregnant female, comprising:
at least two biopotential electrodes for contacting the skin surface of said pregnant female on her abdomen, said electrodes being spacially prearranged to receive surface biopotential signals comprising any of a fetal ECG signal and an EMG signal representative of myometrial activity;
an amplifier network for amplifying said fetal ECG signal and said EMG signal received from said electrodes; and
a power source for powering said monitor device.

2. The apparatus of claim 1, further comprising:
means for separating said ECG signal from said EMG signal.

3. The apparatus of claim 1, further comprising:
means for extracting at least one vital sign.

4. The apparatus of claim 1, further comprising:
means for relaying at least one vital sign to a remote monitor

5. The apparatus of claim 4, wherein said means for relaying a vital sign comprises an electrical cable.

6. The apparatus of claim 4, wherein said means for relaying a vital sign comprises a wireless signal

7. The apparatus of claim 6, wherein said wireless signal comprises any of:
a radio frequency signal, an electrical field, a magnetic field signal, an optical signal, and an ultrasonic signal.

8. The apparatus of claim 1, wherein said vital sign comprises any of uterine contraction, fetal ECG, fetal heart rate, maternal ECG, maternal heart rate, and temperature.

9. The apparatus of claim 1, further comprising:
at least one indicator.

10. The apparatus of claim 9, wherein said at least one indicator indicates any of a vital sign, status of said monitor device, a detected event, status of a detected condition, and an alarm condition.

11. The apparatus of claim 1 further comprising:
at least one electrode placed on the back of the abdomen of said pregnant female.

12. The apparatus of claim 1, said device comprising a patch for adhesive attachment on the skin of said pregnant female.

13. The apparatus of claim 1, wherein said device has a thickness of less than 6 mm.

14. The apparatus of claim 1, wherein said device is shaped to provide surgical access on the abdomen of said pregnant female.

15. The apparatus of claim 1, further comprising:
a wireless sensor for receiving wireless commands from an external instrument.

16. The apparatus of claim 1, wherein said device is disposable after a single use.

17. An integrated patch placed on the body of a pregnant female to obtain one or more vital signs non-invasively, comprising:
at least two sensors contacting the surface of the skin of the pregnant female at the abdomen, said sensors receiving one or more vital signs, one of said sensors comprising at least a pair of biopotential electrodes for receiving an EMG signal representing uterine activity;
an amplifier network for amplifying said vital sign signal from said sensors, and for amplifying said EMG signals received from said biopotential electrodes;
a processor for performing digital signal processing on EMG signals and a vital sign signal;
a power source for powering said monitor patch;
means for extracting at least one vital sign by said processor; and
means for extracting an EMG signal pattern by said processor.

18. The integrated monitor patch of claim 17, wherein said vital sign signal is any of fetal heart rate, fetal ECG, maternal heart rate, maternal ECG, and temperature.

19. The integrated patch of claim 17, wherein said EMG signal pattern comprises any of EMG burst power and EMG burst frequency.

20. The integrated monitor patch of claim 17, further comprising:
means for relaying said at least one vital sign to an external monitor

21. The integrated monitor patch of claim 20, wherein said means for relaying said vital sign comprises a wireless link.

22. The integrated monitor patch of claim 21, wherein said wireless link comprises any of a radio frequency signal, an electrical field, a magnetic field signal, an optical signal, and an ultrasonic signal.

23. The integrated monitor patch of claim 17, further comprising:
at least one indicator activated by said processor.

24. The integrated monitor patch of claim 23, wherein said at lease one indicator indicates any of a vital sign, status of the monitor, status of a detected event, a detected condition, and an alarm condition.

25. The integrated monitor patch of claim 23, wherein said indicator is perceptible by the pregnant female when activated.

26. The integrated monitor patch of claim 23, wherein said indicator comprises a vibratory element for tactile sensing by said pregnant female.

27. The integrated monitor patch of claim 17, wherein said patch is placed on the abdomen of said pregnant female.

28. The integrated monitor patch of claim 17, wherein at least one electrode is placed on back of the abdomen of said pregnant female.

29. The integrated monitor patch of claim 17, said patch is adhesively attached to the skin of said pregnant female.

30. The integrated patch of claim 17, wherein said patch has a thickness of less than 6 mm.

31. The integrated monitor patch of claim 17, wherein said patch is disposable construct that is discarded after a single use.

32. A system for non-invasively monitoring a laboring female, comprising:
a monitor patch adhesively attached and worn on the body of said pregnant female, said patch incorporating biopotential electrodes, corresponding biopotential amplifiers for amplifying an EHG signal and fetal ECG present on the body of said laboring female, a power source for powering said monitor patch, and a wireless transmitter for transmitting data representative of myometrial activity and fetal heart activity;
an interface device incorporating a wireless receiver for receiving said data representing myometrial activity and fetal heart activity sent by said monitor patch, and a signal conditioner for producing an input signal representative of myometrial activity and fetal heart activity to an external monitor; and
an external monitor for receiving said input signal and displaying myometrial activity and fetal heart activity.

33. A method for non-invasive monitoring of a pregnant female, comprising the steps of:
adhesively attaching an integrated patch on the abdomen of said pregnant female, said patch comprising a thin flexible substrate, a biopotential amplifier, biopotential electrodes contacting the skin for receiving composite biopotential signal comprising a composite ECG signal and an EHG signal representative of uterine contractions, a processor, and a power source;
amplifying said biopotential signal obtained from said electrodes with said biopotential amplifier;
extracting myometrial activity data from said EMG signal with said processor; and
extracting fetal heart activity data from said composite ECG signal with said processor.

34. The method of claim 33, further comprising the step of:
computing at least one vital sign.

35. The method of claim 33, further comprising the step of:
activating an indicator by said processor.

36. The method of claim 35, further comprising the steps of:
actuating said indicator when a vital sign parameter exceeds a predetermined limit.

37. The method of claim 35, further comprising the steps of:
integrating said indicator within said patch and;
producing a signal perceptible by the pregnant female wearing said integrated patch.

38. The method of claim 35, further comprising the step of:
providing an said indicator that comprises a vibratory element for producing tactile stimulation perceptible by said pregnant female.

39. The method of claim 33, further comprising the step of:
wirelessly sending data from said integrated patch to an external monitoring device.

40. A method for non-invasive monitoring of a laboring female, comprising the steps of:
adhesively attaching an integrated monitor patch on the abdomen area of said laboring female, said patch comprising a thin flexible substrate, at least two biopotential electrodes contacting the skin of said laboring female at the abdomen for receiving an ECG signal representing fetal heart activity and an EMG signal representing myometrial activity, biopotential amplifiers for amplifying said ECG signal and said EMG signal, and a wireless transmitter;
amplifying said ECG signal and said EMG signal obtained from said biopotential electrodes with said biopotential amplifiers;
sending a wireless signal representing myometrial activity and fetal heart activity with said wireless transmitter to an external monitor; and
displaying myometrial activity and fetal heart activity with said external monitor.
